(19) United States
(12) Patent Application Publication
(10) Pub. No.: US 2013/0039899 A1
(43) Pub. Date: Feb. 14, 2013

(54) METHODS AND DEVICES FOR APPLYING TISSUE SEALANTS AND ADHESIVES

(71) Applicants: Orahn Preiss-Bloom, Zichron Yakov (IL); Ishay Attar, Hof Carmel (IL); Omer Shezifi, Haifa (IL); Yuval Shezifi, Haifa (IL); Golan Salman, Atlit (IL)

(72) Inventors: Orahn Preiss-Bloom, Zichron Yakov (IL); Ishay Attar, Hof Carmel (IL); Omer Shezifi, Haifa (IL); Yuval Shezifi, Haifa (IL); Golan Salman, Atlit (IL)

(73) Assignee: LIFEBOND LTD., Caesarea (IL)

(21) Appl. No.: 13/642,145

(22) PCT Filed: Apr. 20, 2011
(86) PCT No.: PCT/IB2011/051714

§ 371 Date: Oct. 19, 2012

Related U.S. Application Data
(60) Provisional application No. 61/326,211, filed on Apr. 20, 2010.

(51) Int. Cl. A61K 38/45

(52) U.S. Cl. 424/94.5

(57) ABSTRACT

Methods of application and devices thereof for tissue adhesives. The adhesives comprise a plurality of components which are provided separately but which are mixed together to form the adhesive. At least one component is a crosslinkable protein solution and at least one other component is a crosslinking material solution. The devices preferably include a mixing unit, which may include dynamic mixing elements, static mixing elements, or a combination of the two.

### FIELD OF THE INVENTION

The present invention, in at least some embodiments, relates to methods and devices for applying tissue sealants and adhesives, and in particular but not exclusively, to such methods and devices which are useful for applying sealants comprising a mixture of a cross-linkable protein or polypeptide and a non-toxic cross-linking material which induces cross-linking of the cross-linkable protein.

### BACKGROUND OF THE INVENTION

The living human organism contains pressurized fluids, such as blood, urine, lymph, bile, cerebral spinal fluid (CSF), intestinal fluid and air. The liquids are contained in a closed system of vessels, while air is pressurized in the alveolus of the lungs during the inhalation part of the breathing cycle.

The liquid-containing vessel systems can be divided into two categories, high pressure systems and low pressure systems. The arterial blood vessels have the highest pressure, with pulsating pressure in the range of 70-140 mmHG in healthy humans, reaching as high as 220 mmHg in patients suffering from cardiovascular hypertension. Major veins such as the vena cava also show high pulsating pressure, but not as high as that of the arteries. Low pressure systems (having pressure in the range of 10-60 mmHG) include the urinary tract, and systems containing lymph, bile, CSF and intestinal intraluminal content within the gastro intestinal.

Damage to the liquid-containing vessels may occur as a result of surgery, trauma or disease, resulting in leakage of the liquid. Repair of damaged vessels is currently achieved by use of sutures and staples.

Typically, a surgical stapler comprises two stapler arms, one containing one or more lines of multiple staples and a second containing a corresponding structure to bend each of the staples into a closed position. A wide array of stapling devices from different manufacturers is currently available. These vary in staple size, gap width, and staple shape, each having its inherent drawbacks.

The use of stapler devices may result in the leakage of body fluids, such as gastro intestinal content, urine, bile or cerbro spinal fluid (CSF), and in the lungs it can cause pneumothorax.

For some procedures, the use of bare staples, with the staples in direct contact with the patient's tissue, is generally acceptable. The integrity of the tissue itself will normally prevent the staples from tearing out of the tissue and compromising the seam before healing has occurred. In certain circumstances, however, the tissue that is being sealed is too fragile to securely hold the staples in place. In these instances, the tissue will tend to rip at or near the staple lines, slowing healing and often leading to serious complications.

To assist in tissue sealing and adhering, and also hemostasis, various types of sealant or adherent materials may be used, alone or in combination with the above described staples or sutures. However, previously documented low viscosity sealant and adherent materials typically have many drawbacks, a non-exhaustive list of which would include toxicity; insufficient strength; difficulties of application; and non-suitable properties in a surgical environment; among many others.

Efficacious sealant and adherent material with at least one high viscosity component have been documented but previously developed applicator devices, apparatuses, and methods that were developed for use with low viscosity sealants are not equipped to work with higher viscosity materials. Challenges associated with high viscosity sealant and adherent materials include mixing between materials of different viscosity, viscosity variability at different temperatures, non-Newtonian behavior (e.g. viscosity variability in response to stress), higher force requirements, and higher flow resistance.

### SUMMARY OF THE INVENTION

The background art does not teach or suggest suitable devices and methods of application for tissue sealant and adherent materials and, particularly not for tissue sealant and adherent materials where at least one of the components is of high viscosity.

The present invention, in at least some embodiments, overcomes the drawbacks of the background art by providing methods of application and devices thereof for tissue sealant and adherent materials, collectively referred to herein as “tissue adhesives”. Such tissue adhesives may optionally include any hemostatic material, tissue sealant material and tissue adherent material which is a mixture of a cross-linkable protein or polypeptide and a non-toxic cross-linking material which induces cross-linking of the cross-linkable protein. Such an adhesive optionally includes a material which provides an intimate contact and elimination of space between a tissue and a material, including between two tissues. Sealing or adhering therefore includes closure of a tear, wound or puncture in a tissue, and attachment of a material such as a tissue, graft, implant or prosthesis to a tissue. Preferably, the tissue adhesive makes not only direct contact with the surface of the receiving tissue, but also penetrates into the hollows or grooves of the tissue so that mechanical, chemical and/or electrostatic connections or unions or links are formed. Optionally the tissue and the material contact each other only through the sealant, although this is not necessary.

The tissue adhesive preferably has suitable physiological properties to enable it to function well as a medical sealant, adherent and/or hemostatic material, according to at least some embodiments of the present invention. The non-toxic cross-linking material preferably comprises an enzymatic cross-liner. The cross-linkable protein or polypeptide is preferably not fibrin. Therefore the adhesive is preferably an enzyme-crosslinked non-fibrin adhesive.

The non-fibrin adhesive optionally and more preferably has at least the following features, although this list is not intended to be limiting in any way; it is possible that the adhesive has one or more additional features, or even lacks one or more features in the list: no protease inhibitor; single stage enzymatic reaction; can be cofactor independent; can be entirely non blood derived proteins.

According to at least some embodiments of the present invention, optionally the cross-linkable polymer comprises a non-fibrin protein. Optionally the non-fibrin protein comprises gelatin. Optionally the enzyme is selected from the group consisting of calcium dependent or independent transglutaminase, tyrosinase and laccase. Optionally the enzyme comprises microbial transglutaminase. Optionally the composition further comprises a transition point lowering agent for lowering the gelatin transition point. Optionally the enzyme is PEGylated.

According to some embodiments of the present invention there is provided use of the biocompatible medical adhesive, wherein the adhesive further comprises at least one transition point-lowering agent selected from the group consisting of urea and calcium.

Optionally the adhesive further comprises at least one selected from the group consisting of a calcium sequestering agent, a urea sequestering agent, a urea hydrolyzing agent and ammonia scavenging agent.

According to at least some embodiments of the present invention, there is provided a method for mixing a plurality of components to form a tissue adhesive, comprising providing a more viscous liquid and a less viscous liquid, each liquid being a component of the tissue adhesive; adjusting the viscosity of at least one of the more viscous liquid and the less viscous liquid; and mixing the more viscous liquid and the less viscous liquid after adjusting the viscosity to form the tissue adhesive. Optionally, the adjustment of the viscosity is performed by applying shear force to the less viscous liquid or to the more viscous liquid. Also optionally, the adjustment of the viscosity is performed by adding a viscofying agent to the less viscous liquid.

According to at least some embodiments of the present invention, there is provided an apparatus for mixing a plurality of components to form a tissue adhesive, comprising a sponge for receiving a more viscous liquid and a less viscous liquid, each liquid being a component of the tissue adhesive, wherein the sponge is squeezed to cause the liquids to mix and to form the tissue adhesive. The sponge may optionally be the sole device ensuring mixing or as adjunct to another mixing apparatus. The adhesive components may optionally be dispensed into the sponge in various different sequences and locations to improve mixing. The sponge may also optionally be loaded with the two (or more) components of the adhesive in such a configuration that the components are mixed only when the sponge is compressed mechanically, such that the components are dispensed mixed from the sponge, optionally from the borders of the sponge.

For any of the above embodiments, preferably viscosity is adjusted and/or a device is applied to ensure “good mixing” of the components. By “good mixing” it is meant combining or joining of multiple components or ingredients into one homogenous mass, amalgam, or mixture such that any given part of the mixture (ie a sample aliquot of nominal volume drawn from any part of the mixture) contains the same amount and ratio of components. Specifically, the amount of each component coming into contact with or reacting with the amount of other components is preferably uniform across the mixture.

According to at least some embodiments, the tissue adhesive, upon mixing, preferably has viscosity of >500 cP or more preferably >1000 cP at operating room temperature. Optionally, the viscosity of the more viscous component is >1500 cP and more preferably >2000 cP at operating room temperature.

According to at least some embodiments of the present invention, there is provided an apparatus for applying a tissue adhesive, wherein the tissue adhesive comprises a plurality of components, the apparatus comprising a first chamber for receiving a first liquid component of the adhesive, a second chamber for receiving a second liquid component of the adhesive, and a tip in liquid communication with the first chamber and the second chamber, the tip mixing the liquid components to form the adhesive and for extruding the adhesive from the tip for application thereof.

According to at least some embodiments of the present invention, there is provided a method for mixing a plurality of components to form a tissue adhesive, comprising providing a more viscous liquid and a less viscous liquid, each liquid being a component of the tissue adhesive, wherein at least one component comprises a cross-linkable substrate comprising a protein or a carbohydrate, or a combination thereof and at least one component comprises an enzyme capable of cross-linking said substrate; adjusting the viscosity of at least one of the more viscous liquid and the less viscous liquid by applying mechanical or thermal energy to either liquid; and mixing the more viscous liquid and the less viscous liquid after adjusting the viscosity to form the tissue adhesive, such that said cross-linkable substrate is cross-linked by said enzyme to form the adhesive.

Optionally said substrate is selected from the group consisting of gelatin, alginate, chitosan, albumin, collagen, Cellulose derivatives (CMC, MC, HMC, HPMC), Natural gums (Xanthan, Arabic, Guar etc), Whey proteins (lactalbumin, lactoglobulin), dextran, pullulan, curdlan, PVA, PEI, PVP, and PEG.

Optionally said enzyme comprises transglutaminase.

Optionally the more viscous liquid comprises gelatin.

Optionally said applying mechanical energy comprises applying shear force to the less viscous liquid or to the more viscous liquid.

Optionally said applying shear force comprises applying shear force to a non-Newtonian fluid.

Optionally said applying shear force comprises applying shear force to a visco-elastic fluid.

Optionally the method further comprises applying the tissue adhesive only if a viscosity of at least one of said more viscous liquid, said less viscous liquid or said mixture, or a combination thereof, is below a threshold viscosity.

Optionally said threshold viscosity for more viscous component is below a value in the range of 2000-20000 mPa*s or threshold viscosity for mixture of more viscous and less viscous liquids is below a value in the range of 500-10000 mPa*s.

Optionally said threshold viscosity for more viscous component below a value in the range of 2000-10000 mPa*s or threshold viscosity for mixture of more viscous and less viscous liquids is below a value in the range of 500-5000 mPa*s.

According to at least some embodiments, there is provided a method for applying a tissue adhesive, comprising providing a tissue adhesive comprising a plurality of adhesive components, wherein at least one component has high viscosity of about 2000-20000 mPa*s, wherein at least one component comprises a cross-linkable substrate comprising a protein or a carbohydrate, or a combination thereof and at least one component comprises an enzyme capable of cross-linking said substrate; and extruding the tissue adhesive at a predetermined force to apply the tissue adhesive to tissue.

Optionally said substrate is selected from the group consisting of gelatin, alginate, chitosan, albumin, collagen, Cellulose derivatives (CMC, MC, HMC, HPMC), Natural gums (Xanthan, Arabic, Guar etc), Whey proteins (lactalbumin, lactoglobulin), dextran, pullulan, curdlan, PVA, PEI, PVP, and PEG.

Optionally said enzyme comprises transglutaminase.

Optionally the more viscous liquid comprises gelatin.

The method optionally further comprises sealing the tissue with the tissue adhesive after application.

Optionally said predetermined force comprises a linear or non-linear function of sealant viscosity.

The method further comprises predetermining said predetermined force according to a rate of extrusion, wherein said rate of extrusion is determined at least partially according to a rate of cross-linking of the cross-linkable substrate.

Optionally said providing said tissue adhesive further comprises providing an applicator to apply the tissue adhesive, said applicator comprising a body for receiving the tissue adhesive, a tip for extruding the tissue adhesive and a force actuator to control said rate of extrusion from said body through said tip.

Optionally said force actuator comprises a spring.

Optionally said providing said tissue adhesive further comprises providing an applicator to apply the tissue adhesive, said applicator comprising a body for receiving the tissue adhesive, a tip for extruding the tissue adhesive and a downstream flow resistor for controlling extrusion through said tip.

Optionally said downstream flow resistor controls a rate of extrusion from said tip.

Optionally said downstream flow resistor comprises an aperture in said body or said tip, or a combination thereof and wherein said rate of extrusion is determined according to a size of said aperture.

Optionally said rate of extrusion ranges from 0.01 ml to 5 ml per second.

Optionally said cross sectional area of said aperture is variable.

Optionally said area is variable in a range of from 0.1 mm2 to 10 cm2.

Optionally said first and second liquid components, upon mixing, have a viscosity in the range of about 500-20000 mPa*s.

Optionally the method further comprises permitting the tissue adhesive to be extruded only if a viscosity of the tissue adhesive is below a threshold viscosity.

Optionally said threshold viscosity is below a value in the range of 500-10000 mPa*s.

Optionally said size of said aperture determines said threshold viscosity.

Optionally an ambient temperature for applying the adhesive is outside of an optimal performance temperature range of the adhesive.

Optionally a viscosity of sealant or one sealant component is temperature dependent.

Optionally the method further comprises active heating or cooling of sealant.

Optionally said heating or cooling controls a cross-linking rate of the sealant.

Optionally said heating increases said cross-linking rate.

Optionally said active heating or cooling controls a viscosity of at least one component and/or of the adhesive.

Optionally said heat is chemically generated heat, electrically generated heat and/or physically generated heat.

According to at least some embodiments, there is provided an apparatus for mixing a plurality of components to form a tissue adhesive, comprising a sponge for receiving a more viscous liquid and a less viscous liquid, each liquid being a component of the tissue adhesive, wherein said sponge is squeezed to cause the liquids to mix and to form the tissue adhesive.

Optionally said sponge comprises a plurality of pores having a porosity wherein a pore size is in an average diameter range of from 10-1000 μm and wherein average sponge density is in a range of from 0.01-10 g/cm3.

According to at least some embodiments, there is provided an apparatus for applying a tissue adhesive, wherein the tissue adhesive comprises a plurality of components, the apparatus comprising a first chamber for receiving a first liquid component of the adhesive, a second chamber for receiving a second liquid component of the adhesive, a tip in liquid communication with said first chamber and said second chamber, said tip mixing said liquid components to form said adhesive and for extruding said adhesive from said tip for application thereof, and a force actuator for controlling extrusion according to a viscosity of the mixed components wherein said first and second liquid components, upon mixing, have a viscosity of at least a value in the range of 500-10000 mPa*s.

Optionally said force actuator comprises a spring for controlling extrusion from said tip through pressure on said chambers.

Optionally said first liquid component comprises a high viscosity component having a viscosity of at least a value in the range of 1000-20000 mPa*s.

Optionally the apparatus further comprises a downstream flow resistor for controlling extrusion through said tip.

Optionally said downstream flow resistor controls a rate of extrusion from said tip.

Optionally said downstream flow resistor comprises an aperture in said first or second chambers, or said tip, or a combination thereof and wherein said rate of extrusion is controlled according to a cross sectional area of said aperture.

Optionally said cross sectional area of said aperture is variable.

Optionally said area is variable in a range of from area is variable in a range of from 0.1 mm2 to 10 cm2.

Optionally said downstream resistor controls said rate of extrusion in real time.

Optionally said rate of extrusion ranges from 0.01 ml to 5 ml per second.

Optionally the apparatus further comprises a viscosity threshold cutoff mechanism, for permitting the tissue adhesive to be extruded only if a viscosity of the tissue adhesive is below a threshold viscosity.

Optionally said threshold viscosity is below a value in the range of 500-10000 mPa*s.

Optionally said viscosity threshold cutoff mechanism is removed from the apparatus before application of the adhesive.

Optionally the adhesive flows through said viscosity threshold cutoff mechanism during application.

Optionally said tip comprises a flexible tip for application of mixed components, said mixed components forming a high viscosity sealant of about 500-20000 mPa*s wherein tip is of inner diameter between 1.5-5 mm, length between 3-50 cm, and can be bent by 180° C. without impeding flow.

Optionally said body and tip together comprise a sterile fluid path for the adhesive, and wherein one or more chambers receives a non-sterile package comprising a component of said adhesive, such that said sterile fluid path is maintained.

Optionally said non-sterile package or packages is contained within said body during application of the adhesive.

Optionally the apparatus further comprises thermal insulation.

Optionally said thermal insulation comprises a heat sink.

Optionally the apparatus further comprises an active temperature control device for heating or cooling the adhesive prior to and/or during application.

According to at least some embodiments, there is provided surgical apparatus comprising a cutting, stapling, or suturing tool, and a tissue adhesive application system in fluid communication with said tool wherein the adhesive is introduced into the proximal end of the tool from said application system, flows through the body of tool or immediately adjacent to the body of the tool, and is released from the tool.

Optionally the adhesive is released from a distal end of the tool.

Optionally said tool is a trocar, port for single port surgery, or other port for minimally invasive surgery.

Optionally the tool has a secondary port and the adhesive is released from said secondary port.

Optionally said tool is a laparoscopic tool or a surgical stapler.

Optionally said tool is a circular surgical stapler, and adhesive is dispensed through the tip of the stapler anvil shaft, between the tissue surfaces, prior to or during approximation of the tissue surfaces.

According to at least some embodiments, there is provided a method of operation of the apparatus as described herein, comprising approximating two tissue segments for the purpose of attaching the two segments by said tool and releasing said adhesive prior to, during, or immediately following the approximation.

Optionally said approximating is performed manually with a screw-based mechanism or automatically.

According to at least some embodiments, there is provided a method for mixing a plurality of components to form a tissue adhesive, comprising providing a more viscous liquid and a less viscous liquid, each liquid being a component of the tissue adhesive; adjusting the viscosity of at least one of the more viscous liquid and the less viscous liquid; and mixing the more viscous liquid and the less viscous liquid after adjusting the viscosity to form the tissue adhesive, wherein said adjusting the viscosity is performed without alteration of the chemical component(s) of either liquid or the mixture.

Optionally said adjusting the viscosity comprises adding a viscofying agent to the less viscous liquid.

As used herein, “about” means plus or minus approximately ten percent of the indicated value.

Other features and advantages of the invention will be apparent from the following detailed description, and from the claims.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a schematic block diagram of a mixing device according to at least some embodiments of the present invention;

FIG. 2 shows an exemplary mixing tip used for mixing gelatin and mTG (microbial transglutaminase) solutions according to at least some embodiments of the present invention;

FIG. 3A shows a sponge loaded with sealant sits on anvil of circular stapler, as a non-limiting example of a sponge applicator embodiment of the present invention; FIG. 3B shows that as the circular stapler is closed, the sealant is released around the circumference of the sponge;

FIG. 4 shows a non-limiting, exemplary embodiment of a stapler where surgical sealant can be injected into the rear of the stapler and injected out the stapler anvil onto the tissue surface;

FIG. 5A-D are schematic illustrations of an optional embodiment of the present invention for controlling the deployment of a multi-component biological adhesive with a constant force dispensing device; and

FIG. 6A is a perspective view of an optional constant force dispensing device for mixing a plurality of components to form and apply a tissue adhesive in a target tissue, according to an optional embodiment of the present invention;

FIG. 6B is a longitudinal sectional view of the device shown in FIG. 6A according to an optional embodiment of the present invention; and

FIG. 6C provides a close up sectional view showing an optional actuating mechanism depicted in FIG. 6B of the device depicted in FIG. 6A, according to an optional embodiment of the present invention;

FIG. 6D provides a partial exploded perspective view of the device depicted in FIG. 6A showing two components of the device according to an optional embodiment of the present invention;

FIG. 6E provides a partial exploded perspective view of an optional adhesive deploying mechanism according to an optional embodiment of the present invention;

FIG. 6F-G are perspective views of the volume and pressure compensating adaptor according to an optional embodiment of the present inventions.

FIG. 6H-J provide cross-sectional perspective views of optional adhesive application tips according to optional embodiments of the present invention;

FIG. 7A-D show varying views of an optional embodiment of the present invention for a biological adhesive dispenser in the form of a dispensing gun. FIG. 7A shows a perspective side view of the dispensing gun. FIG. 7B provides a cross sectional side view, FIG. 7B shows a close up view of the dispensing gear mechanism; and FIG. 7C provides a partial cross section top down view; and

FIGS. 8 and 9 show various experimental results as described in greater detail below.

### DESCRIPTION OF THE PREFERRED EMBODIMENTS

A non-limiting list of some components of at least some embodiments of an applicator is provided below, with their respective reference numbers:
124 first biological adhesive component vessel;
124a first biological adhesive component
124c first component sterile cover;
124p first component sterile plunger;
126 second biological adhesive component vessel;
126a second biological adhesive component
126c second component sterile cover;
126p second component sterile plunger;
150 adhesive dispensing device;
500 biological adhesive applicator device;
502 outer housing/proximal housing;
501 distal tip housing;
504 mixing element nozzle;
506 adhesive release button;
508 luer tip;
510 inspection window;
512 spring;
514 non-moving plate;
516 connecting plate;
518 actuating apparatus;
520 serrated plunger rod;
522 second plunger rod;
524, 526 chambers;
528 mixing element nozzle connector;
534, 536 two channels;
538 volume/pressure adaptor
540 connecting pin
544 cartridge housing/housing component/medial housing
546, 548 plunger recesses;
550 pinhole;
552 insulating sleeve;
554 coupling aperture;
556 pin distal tip;
558 first component pressure/volume adjusted socket
560 second component pressure/volume adjusted socket
562 flexible tip;
564 elongated tip;
566 mixing and delivery tip
568 first lumen;
570 second lumen
571 mixing lumen
572 mixing element;
574 distal end;
576 spring;
578 plunger;
580 stopper.

The present invention, in at least some embodiments, provides methods of application and devices thereof for tissue adhesives. The adhesives comprise a plurality of components which are provided separately but which are mixed together to form the adhesive. At least one component is a crosslinkable protein solution and at least one other component is a crosslinking material solution. Preferably, the more viscous solution is collagen-derived, and is preferably gelatin, while the less viscous solution is preferably enzymatic, and is preferably transglutaminase. The devices preferably include a mixing unit, which may include dynamic mixing elements, static mixing elements, or a combination of the two. The mixing unit preferably mixes the material in a continuous process as the material is being applied, rather than preparing the entire batch of material at once and then applying it after mixing is completed for the entire batch.

Preferably, static mixing elements are used and the protein solution and crosslinking material solution are introduced to the static mixing unit at a volumetric ratio ranging from 10:1 to 1:10 crosslinking material solution to protein solution. More preferably, the volumetric ratio is 4:1 to 1:4.

In some embodiments of the current invention, the viscosity ratio between the protein solution and crosslinking material solution is greater than 10:1, preferably is greater than 50:1, and more preferably greater than 100:1.

According to at least some embodiments, there is provided a method of application of the tissue adhesive, in which the adhesive is provided as a plurality of separate components. At least one component is a crosslinkable protein solution and at least one other component is a crosslinking material solution. The separate components are then brought together and mixed; if more than two such components are provided, then the components are optionally mixed at once or sequentially, or in combination thereof. The plurality of separate components is preferably provided as a plurality of liquids, in which at least one liquid is more viscous than another liquid and the more viscous liquid is thixatropic (shear thinning).

According to at least some optional embodiments, the method preferably comprises exposing the more viscous liquid to shear force prior to mixing in order to reduce its viscosity and lower the viscosity ratio between the two liquids, thus improving the mixing and increasing the homogeneity of the mixed liquid. The plurality of liquids is then mixed, once the more viscous liquid has been exposed to shear force.

Without wishing to be limited by a single hypothesis, exposing the viscous liquid to shear force both lowers the overall viscosity of the mixture, facilitating greater movement of the two liquids with each other, and lowers the viscosity ratio. A high viscosity ratio between liquids prevents good mixing; thus, lowering the viscosity ratio of the liquids would be expected to increase mixing between the liquids and hence would induce good mixing.

Optionally, the initial viscosity ratio between more viscous and less viscous liquid is >2:1, preferably >5:1, and more preferably >10:1. Optionally, the reduction of more viscous liquid viscosity reduces initial viscosity ratio between more viscous and less viscous liquid by 30%, preferably by 50%, and more preferably by 70%.

Alternatively or additionally, there is provided a method as above for mixing the plurality of components as liquids wherein one liquid is more viscous than another liquid, but wherein the less viscous liquid has the property of shear thickening and is exposed to shear force prior to mixing, in order to increase its viscosity and lower the viscosity ratio between the two liquids, thus improving the mixing and increasing the homogeneity of the mixed liquid. Optionally, the initial viscosity ratio between more viscous and less viscous liquid is >2:1, preferably >5:1, and more preferably >10:1.

Optionally shear force is applied to the less viscous liquid by pushing the less viscous liquid through on or more apertures with cross-sectional area <2 mm², preferably <1 mm², and more preferably <0.6 mm².

Optionally reduction of more viscous liquid viscosity reduces initial viscosity ratio between more viscous and less viscous liquid by 30%, preferably by 50%, and more preferably by 70%.

According to at least some embodiments, the viscosity of the less viscous liquid is increased through the addition of one or more viscosity enhancing agents. Optionally, the viscofying agent is water soluble, preferably, viscofying agent hydroxypropyl methyl cellulose (HPMC) and/or hyaluronic acid (HA).

Again for this embodiment, optionally the initial viscosity ratio between more viscous and less viscous liquid is >2:1, preferably >5:1, and more preferably >10:1.

Also again for this embodiment, optionally enhancing viscosity of the less viscous liquid reduces initial viscosity ratio between more viscous and less viscous liquid by 30%, preferably by 50%, and more preferably by 70%.

Alternatively or additionally, there is provided a method as above for mixing the plurality of components as liquids wherein one liquid is more viscous than another liquid and is incubated at a controlled temperature in order to decrease its viscosity and lower the viscosity ratio between the two liquids, thus improving the mixing and increasing the homogeneity of the mixed liquid. Optionally, the initial viscosity ratio between more viscous and less viscous liquid is >2:1, preferably >5:1, and more preferably >10:1.

Optionally, the less viscous liquid is incubated at the same controlled temperature prior to mixing, such that the less viscous liquid has a lower viscosity decrease than the more viscous liquid, resulting in a net reduction in the viscosity ratio.

Optionally, the less viscous liquid is incubated at a different temperature.

Alternatively, both liquids are incubated at the same controlled temperature but the viscosity ratio does not change.

According to some embodiments, the viscous liquid or both liquids are incubated at a temperature in the range of 18-40° C.

According to some embodiments, the viscous liquid or both liquids are incubated at a temperature in the range of 18-25° C.

According to some embodiments, the viscosity of the mixed sealant varies with temperature and the temperature of the sealant is controlled to achieve viscosity that is optimal for a given clinical application of the sealant. For colorectal and anterior anastomoses, where it is highly undesirable that sealant drips or otherwise leaks from the anastomosis site, sealant viscosity is preferably higher. For low anterior anastomosis, where such movement of the sealant is less undesirable, sealant viscosity may optionally be in the mid-range. For laparoscopic procedures, where sealant needs to flow down a thin scope, viscosity is preferably low.

According to a preferred embodiment of the above, an incubator device is used to control the sealant temperature wherein the device control panel has options for low, mid-range (medium), or high viscosity such that the temperature of the incubator is determined according to the viscosity degree chosen by device operator.

For any of the above embodiments, before being mixed, optionally either liquid can be an emulsion, suspension, or solution. Preferably each liquid is a solution according to at least some embodiments of the present invention. More preferably, the mixture of solutions in itself becomes a solution.

According to at least some embodiments of the present invention, there is provided a method for mixing liquids by pushing them through one or more static mixing elements. Optionally, the static mixer elements are spherical or helical mixing elements. Also optionally, the elements include one or more backflow baffles.

According to at least some embodiments of the present invention, shear force is applied to at least the more viscous liquid (or to a plurality of viscous liquids), by pushing the viscous liquid through on or more apertures with cross-sectional area <2 mm², preferably <1 mm², and more preferably <0.6 mm².

According to at least some embodiments of the present invention, the crosslinkable protein solution and crosslinking material solution form a sealant by being processed through a mixing unit to achieve homogeneity of at least 95% immediately before coming into contact with the target biological system.

Preferably, the crosslinking material solution and crosslinkable protein solution achieve homogeneity of at least 98% after being process through a mixing unit.

Upon mixing of a plurality of liquids, according to at least some embodiments of the present invention, one or more of the liquids being mixed undergoes a polymerization reaction after being mixed with another of the liquids to form a mixture. Preferably, the polymerization reaction causes the mixture to have adhesive properties.

According to other embodiments, there is provided a method for mixing liquids through a matrix, optionally implemented as a sponge, thereby increasing contact between the liquids.

As a non-limiting example, according to some embodiments of the present invention, there is provided a method of mixing of two solutions wherein both solutions are separately injected or otherwise embedded into a porous, compressible matrix (“sponge”) and the matrix is compressed in order to release the homogenously mixed solution.

As another non-limiting example, according to some embodiments of the present invention, there is provided a method of mixing of two substances wherein dry forms of both substances are embedded into a porous, compressible matrix (“sponge”). The matrix is wetted to reconstitute the materials and then compressed in order to release a homogenously mixed solution.

As another non-limiting example, according to some embodiments of the present invention, there is provided a method of uniformly applying a biocompatible material around and/or in the circumference of a circular anastomosis staple-line as detailed in the “contact method” described in Example 5.

Optionally, the biocompatible material is polymerizing.

Optionally, the biocompatible material is a sealant or adhesive.

Optionally, the anastomosis is a colorectal anastomosis.

A device for implementing the above embodiment may optionally comprise a surgical sealant or tissue adhesive applicator, according to at least some embodiments of the present invention, wherein the prepared sealant or adhesive is introduced into a porous, compressible matrix (“sponge”) of defined geometry such that when the sponge is compressed, the sealant is evenly applied to the area surrounding the matrix.

Optionally, the sponge is compressed in the course of operation of a surgical tool such as a circular surgical stapler.

According to other embodiments of the present invention, there is provided a surgical tool intended for cutting, stapling, suturing, or other surgical function that also incorporates a tissue adhesive or surgical sealant application system wherein the sealant or adhesive is introduced into the proximal end of the tool, flows through the body of tool or immediately adjacent to the body of the tool, and is released from the distal end of the tool.

Optionally, the tool is laparascopic tool or a surgical stapler. Also optionally multi-component sealant undergoes mixing within the tool.

With regard to any embodiment relating to application of the tissue adhesive or surgical sealant as described herein, optionally and preferably operation of the surgical tool involves approximating two tissue segments for the purpose of attaching the two segments and the mechanism of tissue approximation drives release of the sealant or adhesive prior to, during, or immediately following the approximation.

Optionally, approximation is done manually with a screw-based mechanism; alternatively, the approximation is performed automatically.

For embodiments in which the surgical tool is a circular surgical stapler, optionally and preferably sealant or adhesive is dispensed through the tip of the stapler anvil shaft, between the tissue surfaces, prior to or during approximation of the tissue surfaces.

According to at least some embodiments, optionally the sealant or adhesive is introduced to surgical tool in non-sterile operating room area in a protected manner that maintains sterility of sealant throughout the application process from injection to application onto the tissue. Also optionally, the sealant is introduced to surgical tool by an external application mechanism during operation (ie not contained in a reservoir prior to operation). Also optionally, the sealant components are mixed in or by the tract leading them from the external reservoir to the distal portion of the tool from which application of the sealant occurs, where the tissue is located.

Attempts have been made to apply tissue adhesives or surgical sealants in the background art, but none solve the problems inherent in the art.

For example, PCT WO/2003/088845 (U.S. Pat. No. 7,517,356) describes a surgical stapler incorporating a reservoir of wound closure material with ducts and a plurality of deployed needles to penetrate the tissue surface and apply the wound closure material. However, the taught device depends on a reservoir of pre-existing wound closure material and thus is not appropriate for a surgical sealant that needs to be mixed either immediately prior to application or during application or for a sealant that undergoes in situ crosslinking. Also the taught device requires application of wound closure material through needles that penetrate a tissue surface, which is highly limiting. Without wishing to be limited by a closed list, the present invention, in at least some embodiments, facilitates mixing prior to application and flowing sealant through body of surgical tool or mixing during application in the surgical tool. Furthermore, as described herein, needles are not required for penetration to tissue in order to apply the tissue adhesive or sealant.

U.S. Pat. No. 7,238,195 describes application of a wound closure material that is activated specifically by the cutting mechanism of a surgical stapler. By contrast, the present invention, in at least some embodiments, does not require activation by the cutting mechanism of such a stapler.

U.S. Pat. No. 5,895,412 describes application of sealant in conjunction with a surgical stapler; however, it is only suitable for a sealant that is activated by heating such that sealant can be applied by having reservoir of sealant that is heated in order to apply it. Thus, the taught device and method for application is irrelevant for a sealant that requires mixing for activation.

According to at least some embodiments of the present invention, there is provided a surgical sealant or tissue adhesive applicator comprised of a disposable or reusable sleeve, mold, or clamp that surrounds the tissue surface to which sealant is being applied. Optionally, the tissue surface is circular and sealant is being applied to circumference, such as the circumference of an intestinal tract or blood vessel. Preferably, the sealant is being applied on top of a staple or suture line.

Optionally, the sleeve, mold, or clamp prevents or reduces dripping of sealant from desired tissue surface.

Optionally, the sleeve, mold, or clamp limits the thickness of sealant layer applied to tissue surface to a thickness in the range of 0.1 mm to 10 mm.

Optionally, the sleeve, mold, or clamp limits the thickness of sealant layer applied to tissue surface to a thickness in the range of 1 mm to 5 mm.

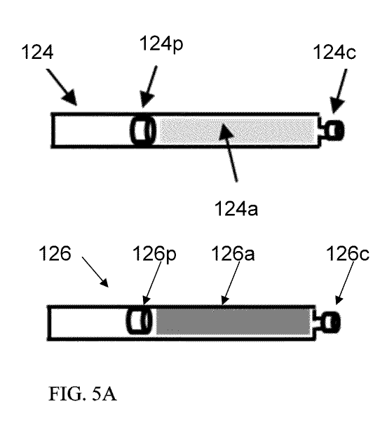

Example 6 shows various non-limiting embodiments of applicators according to the present invention. FIGS. 5-7 show and describe optional embodiments of the present invention for a multi-component biological adhesive dispensing device. Most preferably the optional dispenser embodiments provide for controllably mixing at least two or more adhesive components that when mixed forming a dispensable biological adhesive, according to optional embodiment of the present invention that may be deployed in a target tissue. Most preferably the multi-component dispensing device is a constant force dispensing device comprising at least one or more controls for controlling the adhesive component flow.

### EXAMPLES

Reference is now made to the following examples, which together with the above description, illustrate the invention in a non limiting fashion.

### Example 1

### Achieving Improved Mixing by Shearing Highly Viscous Solution Prior to Mixing it with Less Viscous Solution

Materials:

Low endotoxin gelatin, 275 bloom, type A USP NF porkskin gelatin level 2 (Gelita USA, IA). Microbial transglutaminase solution—purified from Activa TG (Ajinomoto, Japan). Calcium Chloride (Sigma, USA). Urea (Merck, USA). Sodium Citrate (Sigma, USA). Hydroxypropyl Methyl Cellulose (HPMC) (Metolose, Shin-Etsu Chemical Co., Japan). Sodium Acetate (Sigma). Methylene Blue (Sigma).

Solutions:

25% w/w gelatin solution in 4.3M Urea 0.68% (v/v) 1% glycerol, 0.1M Sodium Acetate.

40 enzyme units (EU)/mLmTG (microbial transglutaminase) solution with 1.8% (w/w) HPMC, 0.1M Sodium Citrate, 0.004 mg/ml Methylene blue.

Method:

Gelatin solution was filled into 5 ml syringes. The mTGsolution was filled into 3 ml syringes. Both solutions were then placed at 2-6° C. for 2 hours. After this time, the syringes were placed at 23° C. for 30 min and then below mixing test begun.

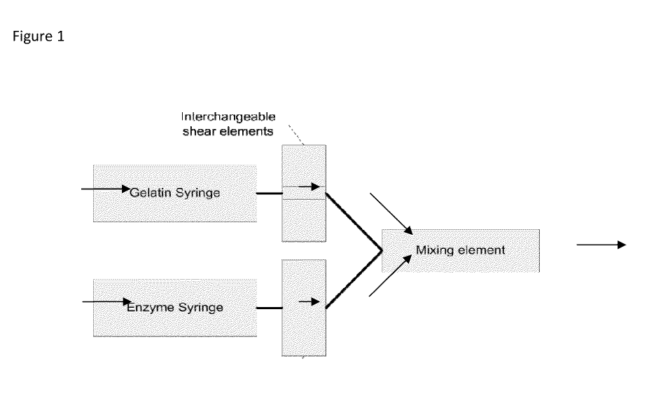

The quality of mixing was tested using a mixing gun and a mixing tip. The gelatin solution was exposed to shear force by pushing it through a small diameter aperture tube (as seen in FIG. 1) prior to mixing. Six different tests were performed; each with a different diameter aperture tube. After passing through the small diameter aperture tube, the gelatin solution came into contact with the enzyme solution and they passed together through a mixing element tube containing 16 5 mm spherical mixing elements. The mixed materials were applied to a white surface to enable accurate visual assessment of mixing.

Mixing was assessed visually by determining if blue enzyme solution and yellowish gelatin solution were separately identifiable after mixing.

FIG. 1 shows a schematic diagram of a mixing apparatus according to at least some embodiments of the present invention. As shown, two syringes are provided, one for each component of the adhesive, which in this specific example are gelatin and an enzyme for cross-linking. An interchangeable shear element is preferably provided for each syringe. After passing through the shear element, the components are preferably mixed in a mixing element, which is preferably a static mixer (for this Example, a static mixer was used).

Results:

| Aperture | Aperture cross | |
|---|---|---|
| diameter | sectional area | |
| (mm) | (mm²) | Results |
| >5 mm | >20 mm | Very difficult mixing. Gelatin comes out |
| | | lumpy. Clearly separate colors, poor mixing. |
| 0.7 mm | 0.38 | Smooth mixing. No differentiation in color. |
| 0.8 mm | 0.50 | Smooth mixing. No differentiation in color. |
| 1.0 mm | 0.79 | Moderately smooth mixing. Visually |
| | | apparent differentiations in color. |
| 1.2 mm | 1.13 | Moderately difficult mixing. Visually |
| | | apparent differentiation in color. |
| 1.4 mm | 1.54 | Very difficult mixing. Gelatin comes out |
| | | lumpy. Clearly separate colors, poor mixing. |

For this case of mixing a thixatropic gelatin solution with a less viscous solution, shearing the gelatin solution (in this example, the higher viscosity component) through a tight aperture, prior to mixing in a static mixer, greatly improved the homogeneity of mixing.

### Example 2

### Example of Achieving Improved Mixing by Adding Viscosity Enhancing Agent to Less Viscous Solution Prior to Mixing it with More Highly Viscous Solution

Materials:

“Gelatin”—275 bloom, type A porkskin gelatin (Gelita USA, IA)

“Na—Ac”—Sodium Acetate trihydrate (Sigma-Aldrich).

“Urea”—98% urea, Mw=60.06 (Sigma-Aldrich).

“Na-Citrate”—Sodium Citrate (Sigma-Aldrich).

“glycerol”—glycerol (Sigma-Aldrich).

“mTG”—Purified mTG (microbial transglutaminase) from ACTIVA TG (Ajinmoto, Japan. Purification included ion exchange (SP sepharose) chromatography and ultrafiltration.

“HPMC”—MethocelHydroxypropyl Methylcellulose (Colorcon).

“MB”—Methylene Blue, MW=319.85 (Riedel-de Haen).

Solutions:

Viscous mTG solution: 40 U/mL mTG in 1.8% (w/w) HPMC in 0.2M Na-Citrate with MB (Methylene Blue).

Non-Viscous mTG solution: 40 U/mL mTG in in 0.2M Na-Citrate with MB (Methylene Blue).

Gelatin solution—25% (w/w) gelatin with 4.3M Urea, 0.68% (v/v) Glycerol in 0.1M Na—Ac pH=6.0.

Method:

Gelatin solution was filled into 5 ml syringes. The mTG solution was filled into 3 ml syringes. Both solutions were then placed at 2-6° C. for 2 hours. After this time, the syringes were placed at 23° C. for 2 hours and then below mixing test begun.

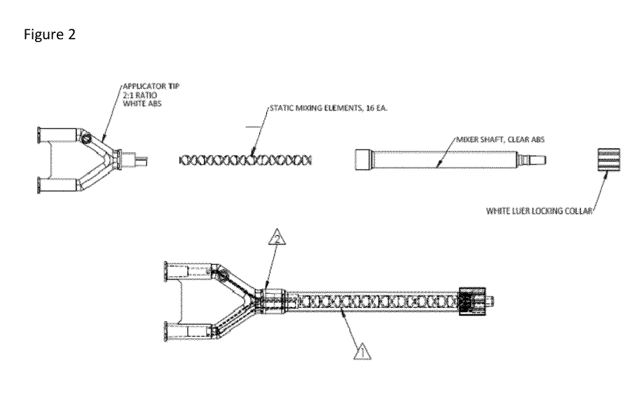

The quality of mixing was tested using a mixing tip with static mixer elements (see FIG. 2 below). The gelatin solution was not exposed to significant shear force prior to mixing. The gelatin solution came into contact with the enzyme solution and they passed together through a mixing element tube containing 16 5 mm spherical mixing elements. The mixed materials were applied to a white surface to enable accurate visual assessment of mixing.

Mixing was assessed visually by determining if blue enzyme solution and yellowish gelatin solution were separately identifiable after mixing.

FIG. 2 shows an exemplary mixing tip used for mixing gelatin and mTG solutions according to at least some embodiments of the present invention. As shown, the tip includes static mixing elements in a mixing shaft.

Results:

| mTG Solution | Results |
|---|---|
| Non Viscous | Liquid mTG solution ran down side of mixer, forming |
| | streaks, and some mTG escaped mixer without |
| | mixing with gelatin. Colors were clearly separate, |
| | indicating poor mixing. |
| Viscous | No differentiation in color. |

For this Example of mixing a viscous gelatin solution with a less viscous mTG (enzymatic) solution, increasing the viscosity of the mTG solution greatly improved the homogeneity of mixing.

### Example 3

### Example of Sealant-Loaded Sponge being Compressed by Operation of Circular Surgical Stapler to Distribute Sealant Around Circumference of Sponge

Methods:

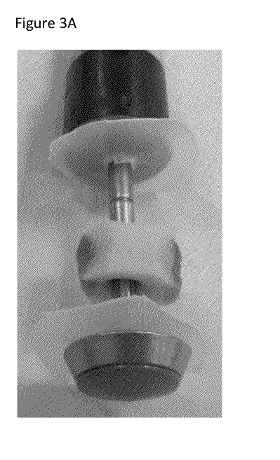

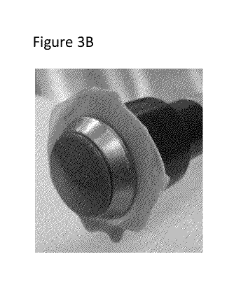

a Sealant as described in Example 1 was well mixed and injected through a 16G cannula into a cylindric section of polyurethane sponge while sponge sat on anvil of circular surgical stapler. When the stapler was closed, the sponge was compressed and loaded sealant was released around circumference of the sponge, as shown in FIG. 3.

FIG. 3A shows a sponge loaded with sealant sits on anvil of circular stapler, as a non-limiting example of a sponge applicator embodiment of the present invention. FIG. 3B shows that as the circular stapler is closed, the sealant is released around the circumference of the sponge.

### Example 4

### Example of Sealant-Loaded Surgical Tool

Methods:

a Sealant as described in Example 1 is well mixed and injected through a luer lock into a surgical tool, such as a surgical stapler for example. When the stapler is closed, loaded sealant is released therefrom.

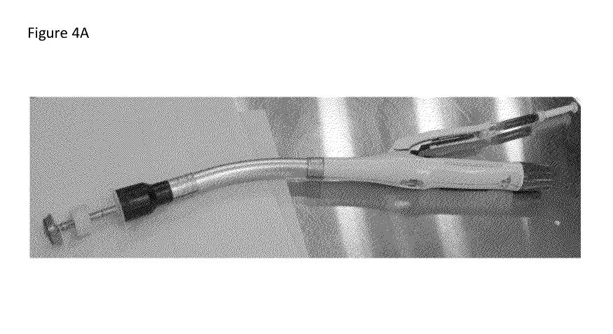

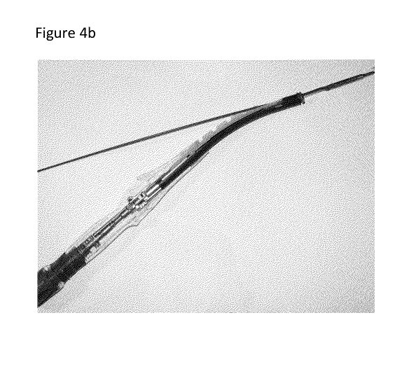

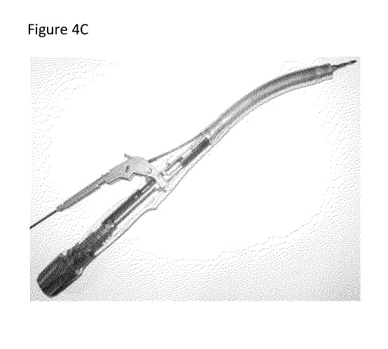

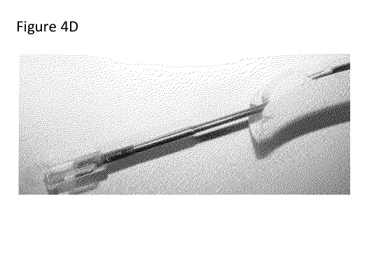

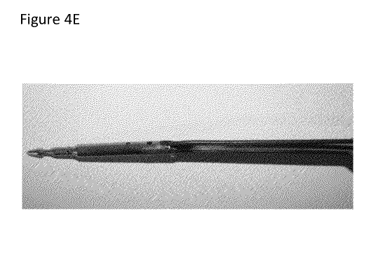

FIG. 4 shows a non-limiting, exemplary embodiment of a stapler where surgical sealant can be injected into the rear of the stapler and injected out the stapler anvil onto the tissue surface. FIG. 4A shows a circular surgical stapler with a built in sealant application system. FIGS. 4B-C show the inner mechanism of a circular stapler with sealant application system. Tubing caries sealant through body of stapler into anvil, where it is dispensed.

FIGS. 4D-E show the rear end of surgical stapler with inlet port for sealant and tip of anvil with outlet tubing for application of sealant through anvil.

### Example 5

### Example of Sealant Application to Circular Anastomosis

Methods:

A 14 day implantation study was undertaken in 6 large white pigs (45+/−3 KG).

In each pig, a midline incision was performed to expose the mid rectum. A 10 mm transaction of the meso was performed along the intended anastomotic line, and a silk 0 knot transmited. A circular stapler (PPC-EEA 28, Covidien, USA) was inserted through the animal's anus. Reaching the appropriate position for the anastomosis, the stapler was opened and held stable and the silk knot was tied around the opened stapler anvil.

Sealant application, of sealant as described in Example 1 that was well mixed, was then performed in one of two ways:
I) Circumferential Method: The stapler was then closed excess string cut. Follow inspection and assurance of the stapler position, an anastomosis was be create in the mid rectum by firing” the staples. The stapler was then opened to 15 mm to bear the staple line. The sealant was then manually applied around the staple line, rotating the intestine to provide access to the entire circumference.
II) Contact Method: Stapler was left open with 3 cm gap between anvil and shaft. ˜3 mL of sealant was then applied in the gap that formed in the tissue between the stapler parts. Stapler was slightly tilted to allow a complete coverage of the tissue in the gap. Stapler was then closed and immediately fired. After ˜10 seconds, trigger was released. When sealant set, after 3 minutes, saline was applied to sealant site. Stapler was then removed.

Three pigs were implanted with sealant using circumferential method and three with contact method.

After implantation, pigs were closed and then monitored for 14 days.

After 14 days, pigs were opened and sealant coverage of anastomotic staple line was macroscopically observed to determine degree of integrity (ie what percentage of staple-line was covered with sealant).

Results

For the three pigs where sealant was applied in the circumferential method, the degree of integrity after 14 days ranged from 60-70%.

For the three pigs where sealant was applied in the contact method, the degree of integrity after 14 days ranged from 90-95%.

### Example 6

### Embodiments of Applicators

Some non-limiting exemplary embodiments of applicators are given below. FIGS. 5-7 show and describe optional embodiments of the present invention for a multi-component biological adhesive dispensing device. Most preferably the optional dispenser embodiments provide for controllably mixing at least two or more adhesive components that when mixed forming a dispensable biological adhesive, according to optional embodiment of the present invention that may be deployed in a target tissue. Most preferably the multi-component dispensing device is a constant force dispensing device comprising at least one or more controls for controlling the adhesive component flow.

FIGS. 5-7 show various non-limiting, exemplary embodiments of applicators for application of sealant/adhesive wherein at least one component has high and the sealant/adhesive is applied using fixed (i.e. preset) force.

The performance of medical and surgical devices can vary greatly depending on user performance as there is high variability between users depending on the specific strength of the user, the geometry of the user's hands, and even the mood of the user. In particular, with surgical sealants and adhesives, it is important that the application be consistent between every procedure to ensure consistent application rate, application coverage, application thickness etc. Unfortunately, with many sealants and particularly with high viscosity sealants, very slight user variability can result in large differences in the layer of sealant that is applied. For example, one surgeon could push out the sealant at a faster rate, perhaps resulting in a thicker or too thick sealant layer being applied to the tissue site; another surgeon could alternatively push out the sealant at a slower rate, perhaps not applying sufficient sealant to the tissue site.

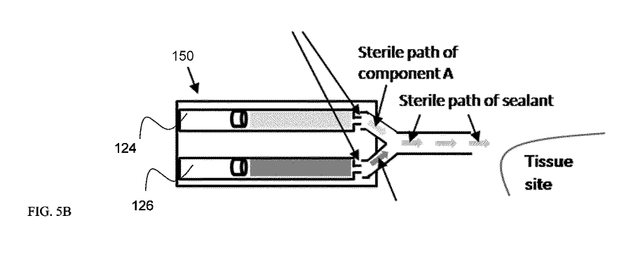

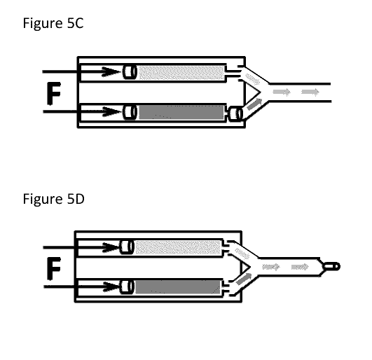

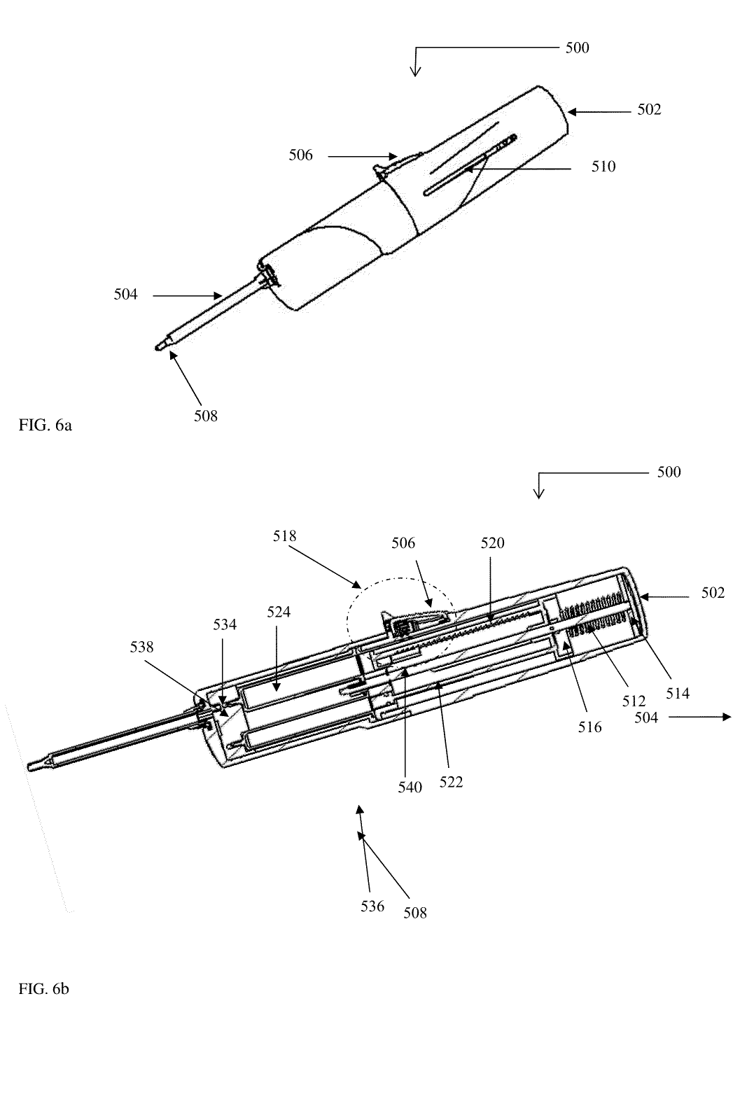

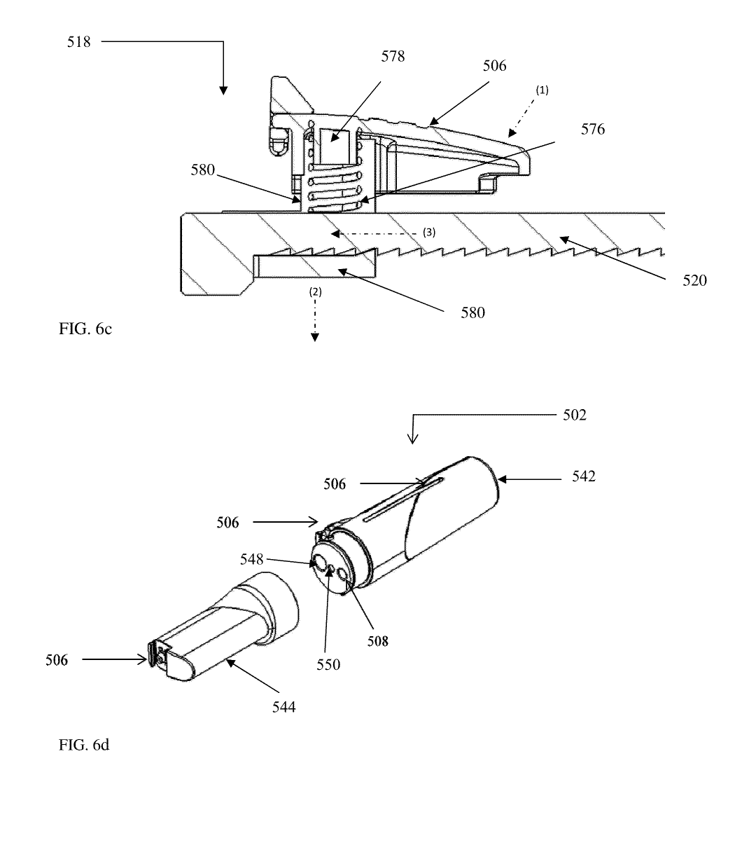

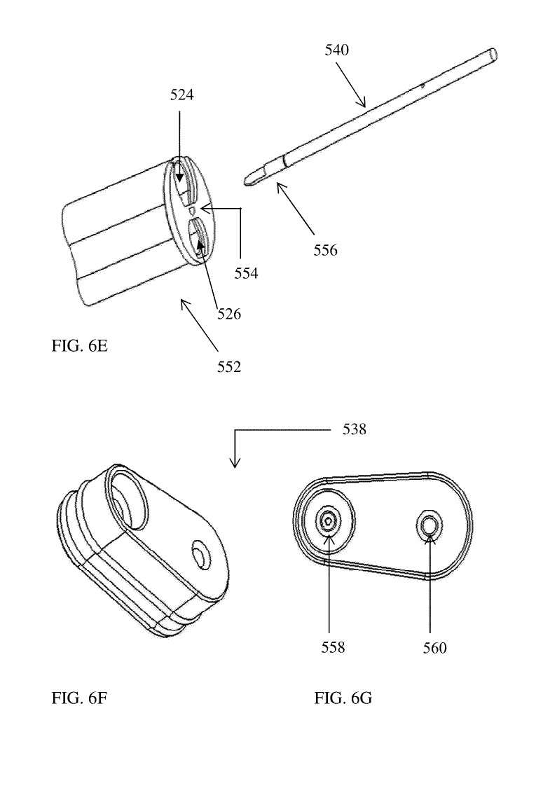

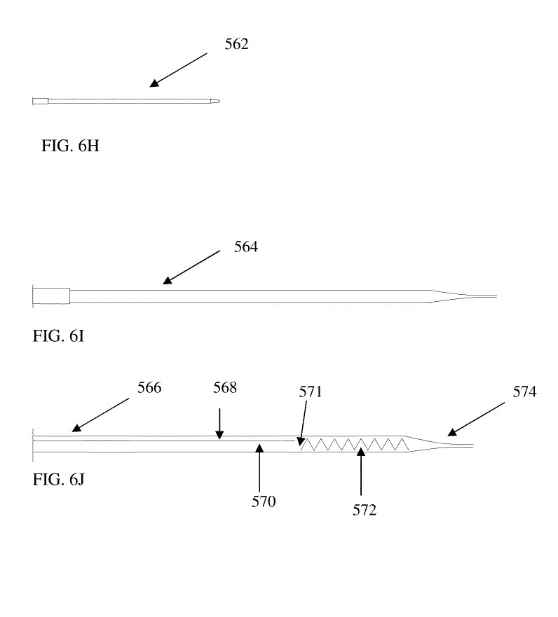

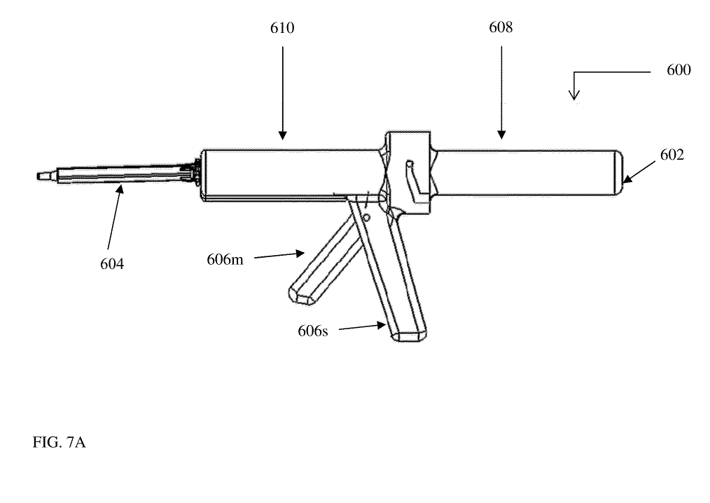

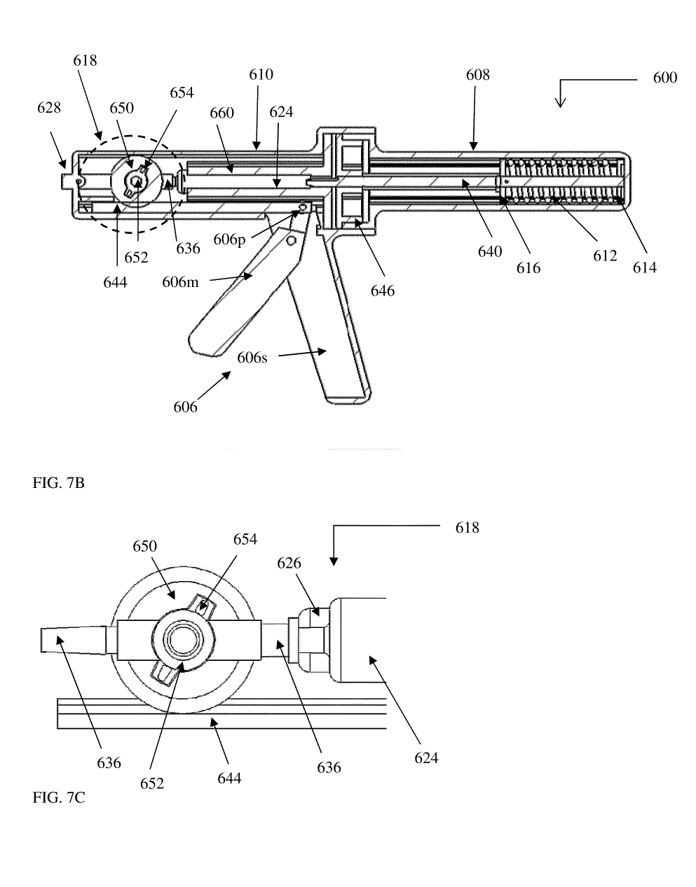

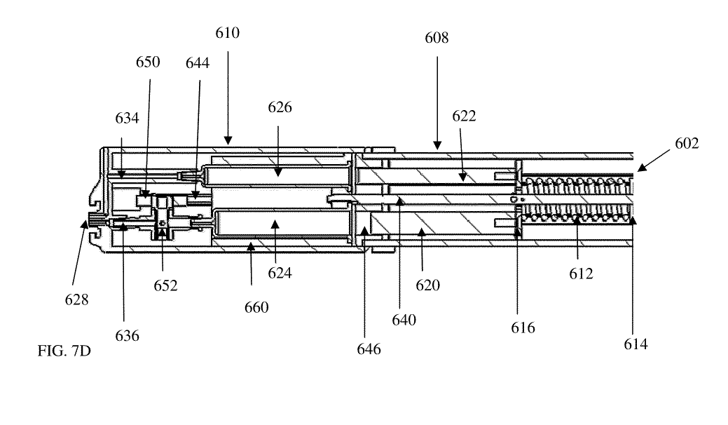

Therefore, there is a need for a new way to reduce the variability between users and improve the consistency of high viscosity sealant/adhesive application. FIGS. 6 and 7 show an applicator that applies a highly viscous sealant/adhesive using a fixed force such that every user will apply the sealant using the same, preset amount of force. This ensures a consistent rate of application and reliable efficacy regardless of user; the fixed force may optionally be applied by a preloaded spring as described in greater detail below.

Also optionally and as shown below, it is possible to control flow rate using a downstream flow resistance mechanism such that resistance could be increased to reduce flow rate or decreased to increase flow rate when equivalent amount of force is applied. If fixed force is applied, optionally varying downstream flow resistance directly changes flow rate.

In particular, when fixed force application is being used, application flow rate would normally be fixed in direct relationship with the amount of fixed force being applied. However, when downstream flow resistance is varied, this resistance will be inversely related to the flow rate. Thus, it is very useful to be able to vary downstream resistance in order to control application flow rate.

Optionally, downstream flow resistance can be varied in real time during the course of sealant/adhesive application.

Also as shown below, the applicator for a variable viscosity sealant/adhesive optionally features a viscosity threshold cutoff. For a sealant/adhesive wherein viscosity of sealant/adhesive is variable such that efficacy of sealant varies with viscosity (i.e. sealant is efficacious below certain viscosity range but not efficacious above certain viscosity range), it is vital that sealant is not applied when it is outside of its efficacious viscosity. However, monitoring sealant viscosity is challenging since traditional methods of measuring viscosity (viscometer, rheometer, etc) are not applicable for sealant that is supplied sterile or aseptic to the surgeon.

However, one embodiment of the herein invention involves an applicator for the variable viscosity sealant where the sealant cannot be expelled from the applicator if it is above a particular viscosity level. This viscosity cut off is a critical mechanism that prevents the sealant from being applied at a viscosity level where it is not efficacious. The viscosity cutoff may optionally be provided with a small diameter aperture such that only sealant at sufficiently low viscosity passes aperture; the cutoff may optionally be in communication with the applicator during actual application of the sealant/adhesive, or alternatively may optionally be removed before such application.

Also as shown below, in at least some embodiments there is provided a flexible tip, which can make clinical application of a surgical sealant much more effective as it allows the surgeon to direct sealant to a precise location without having to manipulate an entire applicator mechanism. However, applying a highly viscous sealant through a flexible tip is challenging since such a flexible tip can significantly increase the resistance of applying a viscous sealant and make this application difficult.

In one embodiment of the herein invention, a tip is incorporated in the applicator that allows for full angle flexibility in application wherein the tip is of dimensions that facilitate application of a highly viscous sealant.

Optionally according to at least some embodiments there is provided an applicator that locks after sealant is inserted into applicator through primary packaging (i.e. syringes, vials, cartridges, etc) such that primary packaging cannot be exposed to sterile surgical environment. In some situations, it is undesirable that the surgical sealant primary packaging be exposed to the sterile surgical environment. For example, if the external of the primary packaging has not undergone terminal sterilization. In these situations, it is desirable that the primary packaging be locked into the applicator to prevent the risk of the packaging being accidentally exposed to the sterile surgical environment.

According to at least some embodiments, the applicator provides a sterile fluid path for application of the sterile sealant from inside the primary packaging. In situations such as above, if the external of the primary packaging has not undergone terminal sterilization, it is undesirable that the surgical sealant come into contact with the external of the primary packaging. In these situations, it is desirable that the sterile sealant or sterile sealant components be able to be dispensed from the primary packaging through a sterile fluid path such that the sealant or sealant components maintain sterility until the sealant is applied to the tissue site.

FIG. 5A provides a schematic illustrative depiction of a first adhesive component 124, and a second adhesive component 126 as shown packaged in a form of a syringe. Most preferably adhesive components housing 124, 126 are provided in a housing that may be associated with a dispensing device 150 (FIG. 5B) for mixing, and dispensing adhesive components 124a, 126a.

Most preferably the individual sealant components 124a,126a have been aseptically filled such that the adhesive components 124a, 126a are sterile, while the sealant component housings, 124, 126 shown in the form of a syringe may be manipulated without compromising the sterility of adhesive components 124a, 126a themselves.

Optionally and preferably the adhesive components 124a, 126a may be sealed within housing 124, 126 at its distal tip by introducing a cap 124c, 126c. Optionally and preferably the adhesive components 124a, 126a may be sealed within housing 124, 126 at its proximal tip by introducing an intrinsic plunger 124p, 126p. Most preferably intrinsic plunger 124p,126p may be associated and/or coupled with a dispensing apparatus provided with an optional dispensers for example as shown in FIGS. 5b, 6a, 7a, as will be described hereinbelow.

FIG. 5B shows an optional embodiment of a dispenser 150 that provides a sterile path for the individual sealant components 124a, 126a, until they are dispensed as a mixture into the target tissue. Dispenser 150 is most preferably provided such that the individual adhesive components 124a, 126a may be extruded from their primary packaging 124, 126, and travel through the applicator 150, and be applied to a tissue site without risk of biological contamination.

Optionally and preferably, dispenser 150 accommodates syringes 124,126 such that the dispenser 150 seals around the sterile tip 124c, 126c, of the syringe 124,126 while enabling sealant components 124a,126a to travel only through a sterile path through the course of extrusion, mixing together, and application to a tissue site.

FIG. 5C shows an optional embodiment of the present invention where a dispensing device 150,600, 500 is provided with dispensing control through one of the adhesive components housing 124,126 and in particular the distal tip 124c or 126c. A more details depiction of such a dispensing device is provided in FIG. 7A-D describing dispenser 600 and in FIGS. 6A-H describing dispenser 500. Most preferably dispensers 150, 500, 600, dispense the adhesive components based on applying a constant force from a proximal end, for example spring 512,612, that is controllably released with a dispensing apparatus 518,618 respectively, as will be described in further details. Optionally and preferably dispensing apparatus 518, 618 provide a form of a flow resistor to regulate the extrusion rate of the sealant 124a, 126a from the applicator 150,500, 600. Most preferably, increasing downstream resistance, with dispensing apparatus 518, 618 lowers extrusion rate and decreasing downstream resistance provides for increasing the extrusion rate

Optionally, varying downstream aperture cross sectional area, for example with adaptor 538, may be used to comprise a downstream flow resistor. For example, for fluid flow wall friction is a significant factor in determining the resistance to flow such that a smaller diameter aperture downstream will introduce more resistance to flow downstream than will a larger diameter aperture, for example adaptor 538. Thus, in an applicator with constant force, from spring 512,612, varying downstream aperture size in effect varies downstream resistance such that larger aperture size will increase extrusion (flow) rate and smaller aperture size will decrease extrusion rate.

Optionally dispenser 150, 500, 600 may be provided with control of the aperture size, for example via varying distal tips, that may be varied over the course of applicator operation, this provides the operator with control to increase or decrease extrusion rate as desired over the course of the application.

FIG. 5C shows a dispensing system with flow resistor aperture downstream of a single component 124. Since application of force is linked, resisting flow of one component is equivalent to resisting flow of entire sealant.

FIG. 5D shows an optional embodiment of the present invention where a dispensing device 150 with dispensing control through the distal tip of the dispenser, for example 501, where the cross-sectional area is controllable.

FIG. 6A shows a perspective view of an optional embodiment of the present invention for a device for mixing a plurality of fluid components to form and delivering/apply/deploy a tissue adhesive in a target tissue area. An optional and preferred embodiment of the present invention provides for mixing two fluid components to form a biological adhesive while providing for the delivery of the biological adhesive to a target tissue so as to optimize adhesive properties of the biological adhesive.

An optional embodiment of the present invention provides for mixing at least two components where each component has individual fluid properties for example viscosity. Most preferably the device 500 according to optional embodiments of the present invention is adapted to mix and deliver the individual components comprising used to formulate the biological adhesive with a single device comprising at least two chambers dedicated to each individual components, and most preferably adapted for their individual viscosity.

Biological adhesive applicator device 500, as shown in FIG. 5A, adapted for deploying and/or delivering a high viscosity adhesives comprising outer housing 502 forming a proximal housing for controlling deployment and/or delivery of the adhesive, a medial housing 544 for storing at least two components that when mixed form the adhesive; and a distal housing 501 forming a tip for mixing the adhesive component and delivering the formed adhesive.

Optionally and preferably outer housing 502 comprises a inspection window 510 provided for visually inspecting, identifying and/or following the progress of the adhesive deployed by device 500. Optionally inspection window 510 may be provided with graduations and/or marking to visually indicate volume of deployed components. Optionally and most preferably window 510 provides an indication of the amount deployed based on the position of a deploying spring 512 (shown and discussed in further detail with respect to FIG. 6B).

Proximal housing 502 most preferably further comprises adhesive release button 506 provided to control the amount of adhesive applied to a target site and/or tissue site. Optionally adhesive release button 506 may be provided in the form of a control button protruding from the external surface of housing 502.

Optionally and preferably distal housing 501 comprising mixing element nozzle 504 and luer tip 508. Optionally distal housing provides for coupling mixing nozzle 504 proximally with medial housing 544 through an appropriate connector 528. Optionally housing 501 may be further coupled with auxiliary distal tips and/or extensions 562,564, 574 (FIG. 5H-J) by coupling an appropriate auxiliary tip and/or device to the distal tip of housing 501 optionally and preferably in the form of luer 508.

Most preferably device 500 is provided as a single device for mixing and delivering an adhesive most preferably a biological adhesive comprising three housing portions namely proximal 502, medial 544 and distal 501 that may be readily coupled and/or decoupled via appropriate connectors, not shown. Optionally any of the proximal housing, medial housing 544 or the distal housing 501 wholly, in part, any combination thereof, may be provided from optional materials so as to render them for at least one or more of single use, wholly disposable, partially disposable, partially multiuse, wholly multiuse, wholly or partially capable of undergoing sterilization, or any combination thereof.

A non-moving plate 514 and the plate 516 hold the spring 512 in a compressed state, the release of the serrated rod 520 enables the spring 512 to apply force on the plate 516 causing it to advance the rods 520 and 522 towards the syringes 524 and 526.

FIG. 6B shows a longitudinal cross section of device 500 as depicted in FIG. 6A, comprising proximal housing 502, medial housing 544, and distal housing 501.

FIG. 6B shows proximal housing 502 comprising; spring 512, a non-moving plate 514 and a plate 516; 518 adhesive deployment actuator assembly; serrated plunger rod 520, second plunger rod 522 and 540 plunger deployment pin. Adhesive deployment assembly 518 includes a deployment button 506 for deploying and/or dispensing the adhesive components as shown in more detail in FIG. 6C. Most preferably deployment assembly 518 may be manually manipulated with deployment button 506 to dispense a biological adhesive at the distal end 508 of device 500. Optionally the deployment 518 may be controlled automatically by wireless, wired, cellular, Bluetooth, or the like communication protocols.

Most preferably compressed spring 512 is disposed between plates 514, 516 to maintain tension in spring 512 provided dispensing the biological adhesive with the assembly 518 and in particular button 506.

Most preferably plungers 520, 522 are provided to dispense respective individual components while simultaneously manipulated and simultaneously controlled with assembly 518 via plunger deployment pin 540 through a plate 516, optionally and most preferably via control button 506.

Optionally and preferably assembly 518 is controllable with button 506, responsible for releasing the serrated plunger rod 520 which is connected to a second plunger rod 522, and plunger pin 540 about plate 516.

FIG. 6C shows a close up view of deployment assembly 518 comprising a spring 576 associated and/or otherwise coupled with plunger 578 having a distal end stopper 580 that corresponds to and engages serrated plunger 520 wherein an edge of stopper 580 corresponds to and engages the serrated edge of plunger 520.

Accordingly pressing button 506, directional arrow 1, compresses spring 576 and plunger 578 to displace stopper 580, as shown by directional arrow 2, to disassociate stopper 580 from an edge of serrated plunger 520 moving plunger 520 distally, shown by directional arrow 3, and then re-associated with a proximal serrated edge of plunger 520. Most preferably serrated plunger 520 is displaced distally due to the distal force acting on plunger 520 formed by the compression of spring 512. Most preferably the distal force of spring 512 is transferred to plunger 520 via through plate 516.

Optionally and preferably serrated plunger 520, plunger 522 and pin 540 are simultaneously displaced distally via plate 516 therein utilizing the compression force of spring 512.

Optionally plungers 520, 522 and pin 540 may be displaced distally via a rotating threaded plunger where for example, external threading about plungers 520 and 522 correspond to internal threading about the inner surface of housing 502 therein mechanically displacing plungers 520, 522 540 relative to housing 502.

Medial housing 544 comprises a first adhesive component chamber for example as shown in the form of a syringe housing 524, second adhesive component chamber for example as shown in the form of a syringe housing chamber 526, volume/pressure adaptor 538 and mixing element nozzle 528. Most preferably volume/pressure adaptor 538 comprises a first channel 534 for receiving the first adhesive component from a first adhesive component chamber 524 and a second channel 536 for receiving the second adhesive component from second adhesive component chamber 526.

Most preferably channels 536 and 534 are directed to a common channel disposed about the mixing element nozzle 528.

Distal housing 501 securely couples and/or is otherwise associated with medial housing 544 over mixing element nozzle 528 wherein mixing element nozzle 504 having luer tip 508 extend from nozzle 528.

FIG. 6D provides a partial exploded perspective view of the device depicted showing the proximal housing 502 separated from medial housing 544, therein exposing the connectors between proximal housing 502 and medial housing 544. Most preferably unidirectional connecting pin 540 couples housing 502 and 544, for example as shown in FIG. 6E, where distal end 556 of pin 540 selectively couples with a corresponding recess disposed about the proximal face of medial housing 544.

Most preferably the distal end 556 of pin 540 passes through the recess 550 disposed about the distal face of housing 502 and couples with aperture 554 dispose about the proximal surface of housing 544 to securely couple housing 502 and 544.

The distal surface of housing 502 further reveals first plunger recess 546 and second plunger recess 548 provided for allowing for the passing of plungers 520 and 522. Most preferably plunger recess 548 is provided accepting and passing serrated plunger 520 while plunger recess 546 is provided for accepting and passing plunger 522. Most preferably recess 546 and 548 correspond with first adhesive component chamber 524 and second adhesive component chamber 526, disposed in medial housing 544, shown in FIG. 5E.

FIG. 6D further shows mixing element nozzle 528 disposed about the distal end of housing 544. Most preferably nozzle 528 provides a platform for mixing the adhesive elements disposed within channels 536 and 534 in the required ratio so as to produce the correct fluid properties for example viscosity of the biological adhesive according to an optional embodiment of the present invention.

FIG. 6E shows insulating sleeve 552 comprising first adhesive component chamber 524 and second adhesive component chamber 526.

Most preferably insulating sleeve 552 provides for insulating or otherwise controlling the temperature of the adhesive components stored in chambers 524 and 526. Optionally sleeve 552 may further comprise materials provided for controlling properties relating to the adhesive components stored in chambers 524 and 526. For example, sleeve 552 may be provided with temperature controlling materials for example heat sink materials, endothermic materials, exothermic materials or the like temperature controlling materials optionally and preferably to control the fluid dynamic and properties of the adhesive components disposed within sleeve 552. Optionally sleeve 552 may be provided with materials for controlling fluid dynamic and properties of the individual adhesive components for example viscosity, that are not solely temperature based. For example, sleeve 552 may comprise heat sink material for example including but not limited to a metal component built into the sleeve.

Optionally sleeve 552 may be provided with a plurality of materials provided for controlling properties relating to the adhesive components stored, for example for individually controlling the properties of the adhesive components in chambers 524 and 526, respectively where the combination may be based on the properties required for the adhesive components. For example, chamber 524 may comprise exothermic materials while chamber 526 may comprise endothermic materials. For example, chamber 524 may comprise endothermic materials while chamber 526 may comprise exothermic materials. For example, chambers 524 and/or 526 may be lined with materials provided for controlling fluid properties, for example, viscosity associated with adhesive components stored in chambers 524 and/or 526.

FIGS. 5F-G show varying perspective views of pressure/volume adaptor 538 provided for controlling the pressure applied to chambers 524 and 526. Optionally and preferably adaptor 538 further provides for controlling the volume released from chambers 524 and 526. The proximal surface of adaptor 538 fits with the distal portion of insulating sleeve 552 while the distal surface of adaptor 538 leads to mixing element nozzle 528, therein providing a continuously fluid passageway through the length of medial housing 544.

Most preferably adaptor 538 comprises at least two or more sockets fluidly connected with the respective chambers of sleeve 552. As shown, a first socket 558 and a second socket 560 may be placed in fluid contact with chambers 524 and 526 of sleeve 552, therein forming the distal end of chambers 524 and 526.

Optionally and preferably sockets 558 and 560 are shaped to receive a container comprising an adhesive component, for example in the form of a syringe tip, Optionally and preferably sockets 558 and 560 are shaped to control and to accommodate different properties and fluid dynamics of the adhesive components utilized with dispenser 500.

Optionally sockets 558 and/or 560 may be shaped and/or otherwise adapted to provide controllable volume dispensing based on pressure applied, for example with plungers 520 and/or 552, to at least two or more adhesive components associated with chambers 524 and 526.

Adaptor 538 is most preferably provided from resilient, compliant and/or elastomeric materials adapted to absorb varying pressures optionally up to about 1000 kPa (kilo Pascal), more preferably adapted to absorb pressures from about 250 kPa to about 1000 kPa and most preferably adapted to absorb pressures from about 360 kPa to about 750 kPa.

Optionally sleeve 552 may be fit with varying adaptor 538 that may be selected based on the adhesive components mixed and/or dispensed with device 500. Optionally sleeve 552 may be fit with varying adaptor 538 that may be selected based on the fluid dynamic properties, for example viscosity, of the adhesive components mixed and/or dispensed with device 500.

FIG. 6H shows a flexible tip 562 that may be connected to the luer tip 508 at the distal end of the mixing element 504, (FIG. 6A). Optionally flexible tip 562, may be provided from pliable, elastic, malleable or the like shape controllable materials to provide for shaping and configuration for applying, deploying and/or delivering the mixed adhesive components to its target site. Optionally tip 562 may be shaped and/or bent to a desired shape to ease the process of applying the material in various, difficult to reach locations.

Flexible tip 562 is preferably adapted for application of mixed components, said mixed components forming a high viscosity sealant of about 500-20000 mPa*s wherein tip 562 is optionally and more preferably of inner diameter between 1.5-5 mm, length between 3-50 cm, and can be bent by 180° C. without impeding flow.

FIG. 6I illustrates an elongated dispensing tip 564 optional and preferably adapted for laparoscopic applications. Optionally, elongated tip 564 may be coupled, connected and/or otherwise securely fixed with mixing element 504 where at least two or more adhesive components are mixed and then passes through to luer 508 and onto dispensing tip 564.

FIG. 6J depicts an optional mixing element and tip 566 that may be utilized for laparoscopic deployment of a multi-component biological adhesive providing for both mixing and dispensing the adhesive. Optionally and preferably tip 566 may be coupled with medial housing 544 via mixing element nozzle connector 528. Tip 566 is preferably a multi-lumen conduit comprising at least two parallel separate lumen 568 and 570 that extend distally to form a single uniform mixing lumen 571, that further extends distally to form the dispensing tip 574. Optionally mixing lumen 571 may be provided with a mixing element 572.

Optionally and preferably tip 566 directly receiving first and second adhesive components dispensed from chambers 524 and 526. Most preferably internal lumen 568 and 570 are adapted to individually receive adhesive components dispensed from chambers 524 and 526 respectively, such that the dispensed adhesive components are not mixed until reaching the mixing lumen 571 optionally comprising a mixing element 572, and deployed at the distal end 574 of tip 566.

FIGS. 7A-D show varying views of an optional embodiment of the present invention for a biological adhesive dispenser in the form of a dispensing gun 600. FIG. 7B provides a cross sectional face on side view, FIG. 7C shows a close up view of the dispensing gear mechanism 618 and FIG. 7D provides a partial cross section top down view. The foregoing description refers to FIGS. 7A-D collectively.

Dispensing gun 600 comprises dispensing handle 606, proximal housing 608, medial housing 610 and distal tip housing 501 (FIG. 6A,6H-J).

Optionally the dispensing gun 600 or any constituents thereof, proximal housing 608, medial housing 610 or the distal housing 501 wholly, in part, any combination thereof, may be provided from varying optional materials so as to render them for at least one or more of single use, wholly disposable, partially disposable, partially multiuse, wholly multiuse, wholly or partially capable of undergoing sterilization, or any combination thereof.

Most preferably proximal housing 608 and medial housing 610 are attached, coupled and/or otherwise securely associated with one another through a coupling pin 640. Dispensing gun 600 is provided for dispensing a biological adhesive, most preferably the biological adhesive is formed by controllably mixing at least two or more fluid adhesive components with dispensing gun 600 to form a biological adhesive while providing for the delivery of the biological adhesive to a target tissue site so as to optimize adhesive properties of the biological adhesive. Most preferably the biological adhesive for dispensing is provided by controllably mixing at least two or more adhesive components stored in a container for example in the form of a syringe that may, optionally and preferably, be disposed in a first and second chamber 624 and 626 respectively.

Most preferably first chamber 624 comprises a first adhesive component container for example provided in the form of a syringe. Most preferably second chamber 626 comprises a second adhesive component container for example provided in the form of a syringe.

Proximal portion 608 comprises a non-moving plate 614 and plate 616 that hold spring 612 in a compressed state. Proximal portion 608 further comprises at least two plungers 620, 622 provided for dispensing adhesive components stored in first chamber 624 and second chamber 626. Most preferably control of plungers 620 and 622 may be provided by dispensing trigger handle 606. Dispensing trigger 606 is linked to stopcock 652, as detailed below, such that depressing trigger 606 opens stopcock 652, allowing syringes to progress as they are no longer blocked by the stopcock 652 in line with the sealant component fluid path.

Distal tip housing 501 as previously described with respect to FIG. 5A-J may attach to be securely coupled with medial housing 610 about the mixing nozzle 628. Mixing element nozzle 628 disposed about the distal end of housing 610 provides a platform for mixing the adhesive components disposed within channels 636 and 634 (FIG. 7D) in the required ratio so as to produce the correct fluid properties for example viscosity of the biological adhesive according to an optional embodiment of the present invention, in passing them through to distal tip 501.

FIG. 7B, shows a side view of dispenser gun 600 that comprises a trigger handle 606 having a moving handle 606m and a stationary handle 606s where moving handle 606m moves with respect to stationary handle 606s to control the volume of biological adhesive dispensed to a target location. Most preferably is provided through mixing nozzle 628 and on to distal tip housing 501, as previously described. Most preferably moving handle 606m is coupled and/or otherwise associated with a rod 644 via pivoting pin 606p such that angular movement of handle 606m causes rod 644 to move linearly back and forth. Most preferably rod 644 is provided with linear gear, threading and/or serration on at least one surface of the rod 644. Preferably threading and/or linear gear and/or serration of a surface of rod 644 engages and/or corresponds to threading and/or gear work of gear 650. Most preferably serrated rod 644 engages with corresponding gear, threading and/or serration with gear 650 to determine, adjust and/or otherwise control the volume of adhesive to be dispensed through mixing nozzle 628 and onto tip housing 501. Most preferably linear movement of rod 644 provides for rotating gear 650, optionally clockwise, to control dispensing of the biological adhesive.

FIG. 7C provides a close up view of dispensing gear apparatus 618 depicting how an adhesive component is dispensed through to mixing nozzle 628. Optionally and preferably dispensing gear apparatus 618 comprises gear 650 that is concentrically coupled to bore stopcock 652 such that the rotation of gear 650 cause rotation of bore stopcock 652 in the direction of motion of gear 650. Most preferably gear 650 is rotated with the triggering motion of handle 606m, as previously described.

Optionally and preferably bore stopcock 652 is provided with at least one internal passageway channel 654. Optionally stopcock may be provided with at least two or more internal passageway channels 654. Most preferably when channel 654 is aligned and overlaps with channel 636, adhesive materials stored in syringe channels 624 are allowed to flow therethrough. Optionally stopcock 652 may be controllably aligned and or selectively adjusted to control the amount of and/or timing of adhesive components release through channel 654. For example, stopcock 652 may be adapted to release a first adhesive component at a fast rate while a second adhesive component at a slower rate, for example based on the number of stopcock channel 654 and/or stopcock 652 timing and/or alignment.

FIG. 7D showing a top down cross section of a partial segment of the medial housing 610 and proximal housing 608, comprising compressed spring 612 that is backed by the plate 614 which is attached to the outer cover 602, the spring 612 pushes the plate 616 when the stopcock bore 654 overlaps with the tunnel 636. Most preferably rods 620 and 622 to advance through the inner cover 646 to the chambers 624 and 626 inside the sleeve 660.

Most preferably sleeve 660 comprising first adhesive component chamber 524 and second adhesive component chamber 626. Optionally and preferably sleeve 660 may be provided as an insulating sleeve. Insulating sleeve 660 may provide for insulating or otherwise controlling the temperature of the adhesive components stored in chambers 624 and 626. Optionally sleeve 660 may further comprise materials provided for controlling properties relating to the adhesive components stored in chambers 624 and 626. For example, sleeve 660 may be provided with temperature controlling materials for example heat sink materials, endothermic materials, exothermic materials or the like temperature controlling materials optionally and preferably to control the fluid dynamic and properties of the adhesive components disposed within sleeve 660. Optionally sleeve 660 may be provided with materials for controlling fluid dynamic and properties of the individual adhesive components for example viscosity, that are not solely temperature based. For example, sleeve 660 may comprise heat sink material for example including but not limited to a metal component built into the sleeve.

Optionally sleeve 660 may be provided with a plurality of materials provided for controlling properties relating to the adhesive components stored, for example for individually controlling the properties of the adhesive components in chambers 624 and 626, respectively where the combination may be based on the properties required for the adhesive components. For example, chamber 624 may comprise exothermic materials while chamber 626 may comprise endothermic materials. For example, chamber 624 may comprise endothermic materials while chamber 626 may comprise exothermic materials. For example, chambers 624 and/or 626 may be lined with materials provided for controlling fluid properties, for example, viscosity associated with adhesive components stored in chambers 624 and/or 626.

Optionally and preferably the adhesive components in each syringe chamber 624, 626 may be extruded to the respective dispensing channels 634 and 636 which lead in parallel to the mixing element nozzle 628. Optionally and preferably control of the amount of the adhesive components extruded is controlled by the overlap percent of the stopcock bore 652 and the channel 636.

### Example 7

### Viscosity Measurements

This example establishes the standard method used to establish the viscosity values provided throughout the application.

Furthermore, the viscosity measurements of crosslinkable substrate (gelatin) and crosslinker (enzyme solution) are described to demonstrate that a very significant viscosity gap can exist between a substrate solution and crosslinker substrate. Such a viscosity gap makes it difficult to mix the solutions. In this case, heating the substrate solution greatly decreases its viscosity and facilitates improved mixing of the two components.

Materials:

Low endotoxin gelatin, 275 bloom, type A USP NF porkskin gelatin (Gelita USA, IA). Microbial transglutaminase solution, purified from Activa TG (Ajinomoto, Japan). Calcium Chloride (Sigma, USA). Urea (Merck, USA). Sodium Citrate (Sigma, USA). Hydroxypropyl Methyl Cellulose (HPMC) (Metolose, Shin-Etsu Chemical Co., Japan). Sodium Acetate (Sigma). Methylene Blue (Sigma), Silicon Oil (Poly-Alpha-Olefin, Cannon instrument company, PA USA)

Gelatin solutions: 25% w/w gelatin solution in 4.3M Urea 0.68% (v/v) 0.1M Sodium Acetate.

Crosslinker solution: 60 enzyme units (EU)/mL mTG solution with 1.5% (w/w) HPMC, 0.1M Sodium Citrate, 0.004 mg/ml Methylene blue.

Methods:

All viscosity values throughout this application are based on viscosity measurements performed using a AR1500ex (TA Instruments, New Castle, Del.) Air Bearing Rheometer System with 40 mm 1 degree cone geometry and computer controlled peltier plate. All viscosity measurements were performed at shear rate of 1 s⁻¹.

Viscosity value standard was established by measuring silicon oil at above conditions at temperature of 25° C.

Gelatin solution viscosity was tested at 19° C. and at 25° C., each for 5 minutes at constant shear rate of 1 s⁻¹. Crosslinker solution viscosity was tested at 19° C.

Results:

Viscosity of silicon oil at 25° C. was measured to be 3018 mPa*s (1 cP=1 mPa*s)

Viscosity of gelatin solution at 19° C. was 16 Pa*s after 5 minutes. Viscosity of crosslinker solution at 19° C. was 2.5 Pa*s after 5 minutes.

Viscosity of gelatin solution at 25° C. was 2.1 Pa*s after 5 minutes.

### Example 8

### Application of Shear Force to Modify Viscosity of Sealant Component

This example demonstrates that the viscosity of a high viscosity component of a sealant can be reduced by applying shear force.

Methods:

Gelatin solution was prepared as in example 7, above. Viscosity of gelatin solution at 18° C. was measured for 2 minutes using measurement conditions and equipment described in example 7, above. Shear force of 100 s⁻¹ was then applied to gelatin solution for 30 seconds and viscosity measurement was repeated.

Results:

After 2 minutes at 18° C., gelatin solution viscosity was 346 Pa*s. After 30 seconds of shearing at 100 s⁻¹, viscosity measurement was 30 Pa*s.

### Example 9

### Fixed (Predetermined) Force to Apply Sealant

This example demonstrates that predetermined, fixed force can be used to apply a multi-component sealant. It further demonstrates that a sealant component viscosity (as demonstrated in example 7, gelatin solution viscosity changes with temperature) increases the amount of force required to apply sealant component in a given amount of time and that these considerations can help determine the choice of a particular amount of force.

Methods

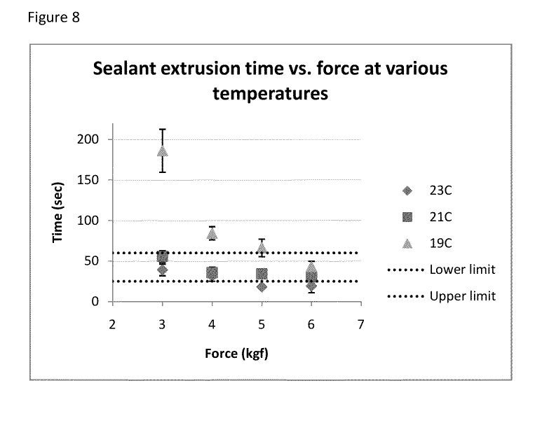

Gelatin solution, as in example 7, was filled into 5 ml syringes. The enzyme solution (crosslinker solution as in example 7) was filled into 3 ml syringes. Both solutions were then placed at 2-6° C. for 24 hours. After this stabilization time, the syringes were placed at 23° C. water bath for 60 min until gelatin component was fully thawed and stabilized. The gelatin and enzyme syringes were then placed at controlled dry environment of 16±1° C. while gelatin temperature was measured using a thermometer (TES-1316, TES Electrical, Taiwan) and T-type thin thermocouple (Elcon LTD, Israel) inserted into the syringe. The two syringes were exposed to a 16±1° C. environment for 0, 15, or 30 min, where the gelatin temperature reached 23, 21, or 19° C., respectively. The two syringes were then loaded into a manual applicator apparatus fixed vertically. A constant force was applied to extrude the two components through the apparatus using 3, 4, 5, or 6 kg weights. After pressed, the components were mixed in a static mixing element (cat #7701836 by TAH, USA) and secreted through a 14G cannula (Suruflon, Suru, India). The time to complete material extrusion was measured for each of the 12 gelatin temperature and extrusion force combinations. Five repetitions were made for each combination, and the mean±SD values are displayed in FIG. 8.

Results

The forces range 4-6 kg yielded extrusion rates of 30-60 s (defined here as the desired extrusion time) when appropriately determined force was applied to gelatin solution at appropriate temperatures.

### Example 10

### Thermal Insulation Test

This example demonstrates that thermal insulation, with or without a heat sink, can maintain the temperature of a surgical sealant component in a desired range for longer than temperature is maintained without thermal insulation.

Gelatin solution, as in example 7, was filled into 5 ml syringes. The enzyme solution (crosslinker solution in example 7) was filled into 3 ml syringes. Both solutions were then placed at 2-6° C. for 24 hours. After this time, the syringes were placed in 23° C. water bath for 60 min until gelatin component was fully thawed and stabilized. The gelatin and enzyme syringes were then loaded into an insulating sleeve and the surrounding cartridge (604 in drawings) which were placed at controlled dry environment of 16±1° C. while gelatin temperature was measured and logged by a thermometer (TES-1316, TES Electrical, Taiwan) in 1 min intervals using a T-type thin thermocouple (Elcon LTD, Israel) inserted into the syringe until reaching 19° C. This procedure was repeated 3 times for each of the following:

Insulating sleeve: Plastic sleeve

Heat sink: Sleeve with two metal cylindrical inserts, 3 mm diameter, in the length of the entire sleeve.

Control: No sleeve or insulation element

Results

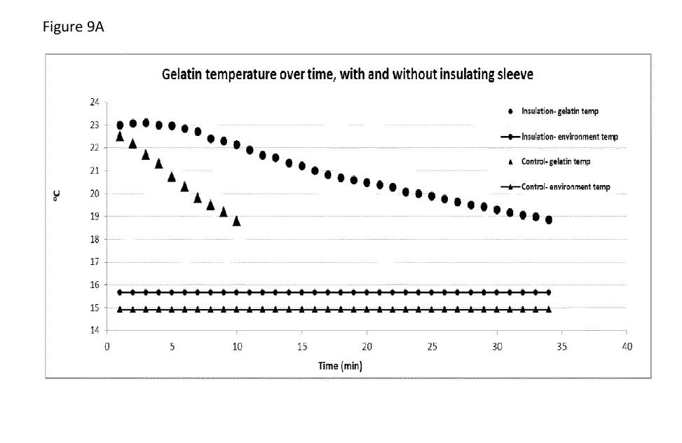

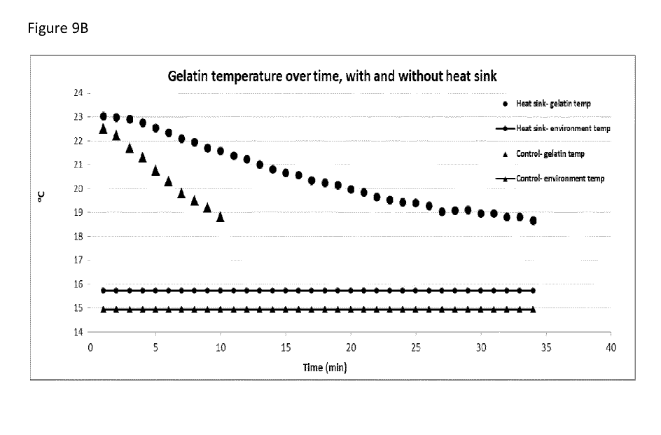

Time to reach 19° C., bottom limit of desired efficacious sealant component range, was 33 min with an insulating sleeve, 28 min with a heat sink sleeve, and 8 min for Control (no insulation). Results are shown in FIGS. 9A and 9B.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for mixing a plurality of components to form a tissue adhesive, comprising providing a more viscous liquid and a less viscous liquid, each liquid being a component of the tissue adhesive, wherein at least one component comprises a cross-linkable substrate comprising a protein or a carbohydrate, or a combination thereof; wherein said substrate is selected from the group consisting of gelatin, alginate, chitosan, albumin, collagen, cellulose or cellulose derivatives (CMC, MC, HMC, HPMC), natural gums (Xanthan, Arabic, Guar), whey proteins (lactalbumin, lactoglobulin), dextran, pullulan, curdlan, PVA, PEI, PVP, and PEG; and at least one component comprises an enzyme capable of cross-linking said substrate; adjusting the viscosity of at least one of the more viscous liquid and the less viscous liquid by applying mechanical or thermal energy to either liquid; and mixing the more viscous liquid and the less viscous liquid after adjusting the viscosity to form the tissue adhesive, such that said cross-linkable substrate is cross-linked by said enzyme to form the adhesive; further comprising applying the tissue adhesive to the tissue only if a viscosity of at least one of said more viscous liquid, said less viscous liquid or said mixture, or a combination thereof, is below a threshold viscosity; wherein said threshold viscosity for more viscous component has a value in the range of 2000-20000 mPa*s or threshold viscosity for mixture of more viscous and less viscous liquids has a value in the range of 500-10000 mPa*s.

2. (canceled)

3. The method of claim 1, wherein said enzyme comprises transglutaminase.

4. The method of claim 2, wherein the more viscous liquid comprises gelatin.

5. The method of claim 1, wherein said applying mechanical energy comprises applying shear force to the the more viscous liquid.

6. The method of claim 4, wherein said applying shear force comprises applying shear force to a non-Newtonian fluid.

7. The method of claim 4, wherein said applying shear force comprises applying shear force to a visco-elastic fluid.

8. (canceled)

9. (canceled)

10. The method of claim 1, wherein said threshold viscosity for more viscous component below a value in the range of 2000-10000 mPa*s or threshold viscosity for mixture of more viscous and less viscous liquids is below a value in the range of 500-5000 mPa*s.

11. The method of claim 1, wherein said applying the adhesive to the tissue comprises extruding the tissue adhesive at a predetermined force to apply the tissue adhesive to tissue.

12. (canceled)

13. (canceled)

14. (canceled)

15. The method of claim 1, further comprising sealing the tissue with the tissue adhesive after application.

16. The method of claim 8, wherein said predetermined force comprises a linear or non-linear function of sealant viscosity.

17. The method of claim 10, further comprising predetermining said predetermined force according to a rate of extrusion, wherein said rate of extrusion is determined at least partially according to a rate of cross-linking of the cross-linkable substrate.

18. The method of claim 1, wherein said applying said tissue adhesive further comprises providing an applicator to apply the tissue adhesive, said applicator comprising a body for receiving the tissue adhesive, a tip for extruding the tissue adhesive and a force actuator to control said rate of extrusion from said body through said tip.

19. The method of claim 12, wherein said force actuator comprises a spring.

20. The method of claim 1, wherein said applying said tissue adhesive further comprises providing an applicator to apply the tissue adhesive, said applicator comprising a body for receiving the tissue adhesive, a tip for extruding the tissue adhesive and a downstream flow resistor for controlling extrusion through said tip.

21. The method of claim 14, wherein said downstream flow resistor controls a rate of extrusion from said tip.

22. The method of claim 14, wherein said downstream flow resistor comprises an aperture in said body or said tip, or a combination thereof and wherein said rate of extrusion is determined according to a size of said aperture.

23. The method of claim 16, wherein said rate of extrusion ranges from 0.01 ml to 5 ml per second.

24. The method of claim 16, wherein said aperture comprises a variable cross sectional area.

25. The method of claim 18, wherein said area is variable in a range of from 0.1 mm² to 10 cm2.

26. The method of claim 1, wherein said more viscous liquid and less viscous liquid, upon mixing, have a viscosity in the range of about 500-20000 mPa*s.

27. (canceled)

28. (canceled)

29. The method of claim 16, wherein said size of said aperture determines said threshold viscosity.

30. The method of claim 1, wherein an ambient temperature for applying the adhesive is outside of an optimal performance temperature range of the adhesive.

31. The method of claim 10, wherein said viscosity of said sealant is temperature dependent.

32. The method of claim 1, further comprising active heating or cooling of a sealant.

33. The method of claim 24 wherein said heating or cooling controls a cross-linking rate of the sealant.

34. The method of claim 25, wherein said heating increases said cross-linking rate.

35. The method of claim 1, further comprising active heating or cooling to controls a viscosity of at least one component and/or of the adhesive.

36. The method of claim 27, wherein said heat is chemically generated heat, electrically generated heat and/or physically generated heat.

37-73. (canceled)
